(19) United States
(12) Patent Application Publication
(10) Pub. No.: US 2004/0132817 A1
(43) Pub. Date: Jul. 8, 2004

(54) Use of an histone deacetylase inhibitor for the treatment of diseases associated with an hpv infection

(76) Inventors: Patrick Finzer, Mannheim (DE); Frank Rosl, Neuhofen (DE); Harald Zur Hausen, Waldmichelbach (DE)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/474,628

(22) PCT Filed: Apr. 10, 2002
(86) PCT No.: PCT/EP02/04004

(30) Foreign Application Priority Data
Apr. 10, 2001 (EP) 01108923.3

(51) Int. Cl. A61K 31/19

(52) U.S. Cl. 514/557; 514/575

(57) ABSTRACT

Described is the use of histone deacetylase inhibitors for the treatment of a disease, preferably cervical cancer, associated with an HPV infection. Particularly useful inhibitors are sodium butyrate, phenylbutyrate and trichostatin A.

### RELATED APPLICATIONS

This application is a national stage filing of international application PCT/EP02/04004, filed Apr. 10, 2002, which claims priority to European application No. EP01108923.2, filed Apr. 10, 2001, each incorporated by reference in their entirety.

### I. FIELD OF THE INVENTION

The present invention relates to the use of histone deacetylase inhibitors for the treatment of a disease, preferably cervical cancer, associated with an HPV infection. Particularly useful inhibitors are sodium butyrate, phenylbutyrate and trichostatin A.

### II. BACKGROUND OF THE INVENTION

Carcinoma of the uterine cervix (cervical cancer, CC) is the second most common cancer in women worldwide and the first in developing countries. CC develops through premalignant intermediate stages of increasing severity known as cervical intraepithelial neoplasm (CIN) grades 1-3, the latter leading to the development of invasive cancer in about 50% of cases over a period of 1-2 decades. More than 11% of the global cancer incidence in women is due to human papillomavirus (HPV) infections. Infection with HPV-types 16 and 18 has been associated with the development of CIN and CC, with HPV genotype 16 being the most prevalent viral type to infect the cervix. The E6 and E7 proteins encoded by these HPV types are thought to be involved in the pathogenesis of CC by inducing abnormal cell proliferation. Expression of E6 and E7 is consistently detected in tissue and tumor cells from HPV-associated CCs. Furthermore, the E6 and E7 genes from HPV types 16 and 18 are sufficient for transformation of epithelial cells in culture.

There is increasing evidence that the E6 and E7 viral oncogenes encoded by HPV types 16 and 18 may be effective targets for tumor rejection by the host and that a therapy might be based on inactivation of said proteins or inhibition of expression of the corresponding genes. Unfortunately, the different strategies employed so far for therapy gave only discouraging results. Theoretically, a treatment might be achieved by methods like, e.g., the suppression of the expression of viral oncogenes using antisense-approaches or interference of specific protein interactions between cellular proteins and viral oncogenes using aptameres. However, at present it is unclear whether these strategies will ever work, since, e.g., as regards antisense-based approaches it is entirely open as to whether in vivo any effect by oncogene suppression can be obtained and with respect to the use of aptamers it is so far entirely unclear as to whether they are even capable of entering an oncogene-positive cell. Moreover both approaches are time and cost consuming, thus, presumably not generally suitable for therapy.

Therefore, it is the object of the present invention to provide means allowing the treatment of diseases associated with an HPV infection.

### III. SUMMARY OF THE INVENTION

Described is the use of histone deacetylase inhibitors for the treatment of a disease, preferably cervical cancer, associated with an HPV infection. Particularly useful inhibitors are sodium butyrate, phenylbutyrate and trichostatin A.

### IV. BRIEF DESCRIPTION OF THE DRAWINGS

FIG. 1: Sodium butyrate (NaB) inhibits G1 to S phase transition in HeLa cells without affecting viral oncogene expression:

(A) Upper part: representative flow-cytometric profile of HeLa cells after treatment with 6 mM sodium butyrate (NaB) for 16 hours. “Contr.”: untreated control. The different phases of the cell cycle are indicated (G1, S, G2, respectively). Lower part: quantification of the number of cells during the cell cycle (means of three independent experiments).

(B) Northem blot analysis. 5 μg of RNA were separated in a 1% agarose gel. The filter was consecutively hybridized with a HPV18 and GAPDH probe. The positions of the 28S and 18S ribosomal RNA are indicated.

(C) Western blot analysis. 75 μg of total cellular protein was loaded on a 12% SDS-PAGE gel. After electrotransfer, the filters were consecutively incubated with HPV18 E7 and a monoclonal actin antibody. (−): untreated cells; (+): treated with 6 mM sodium butyrate for 16 hours.

FIG. 2: Effects of sodium butyrate on the expression of G1 cyclins and cyclin dependent kinases (cdks).

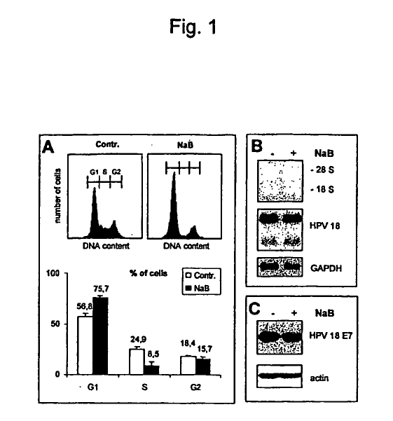

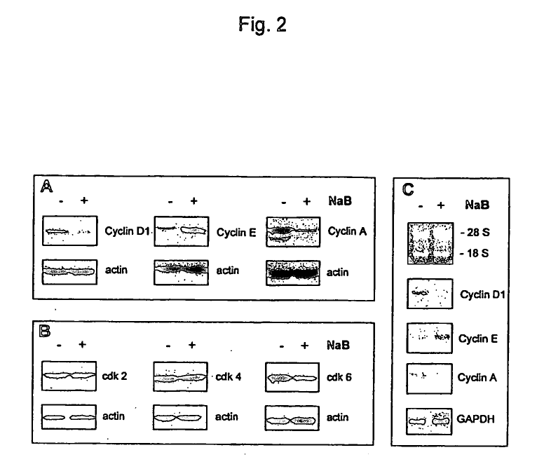

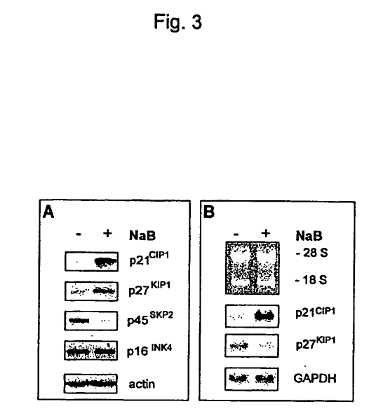

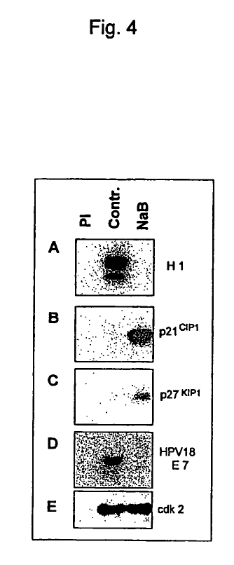

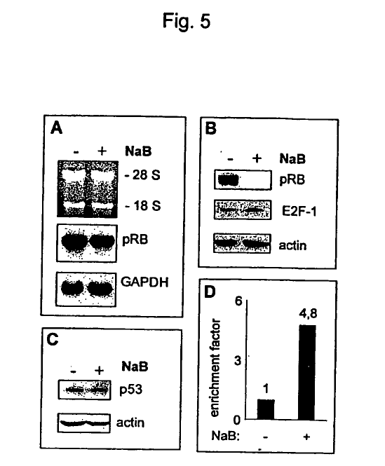

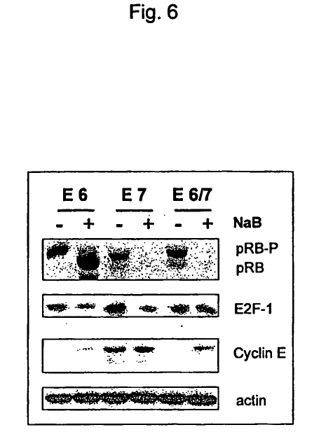

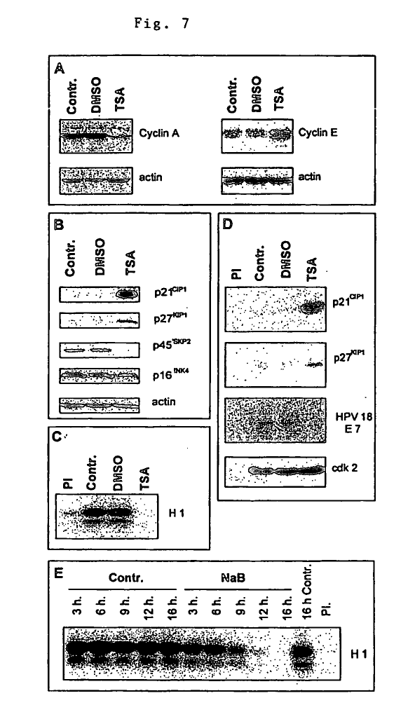

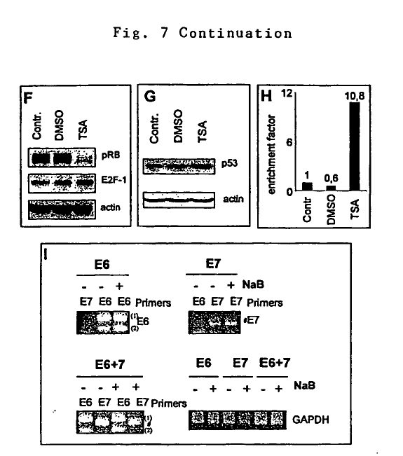

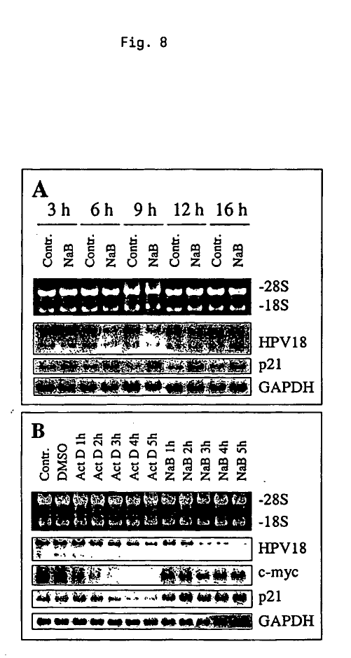

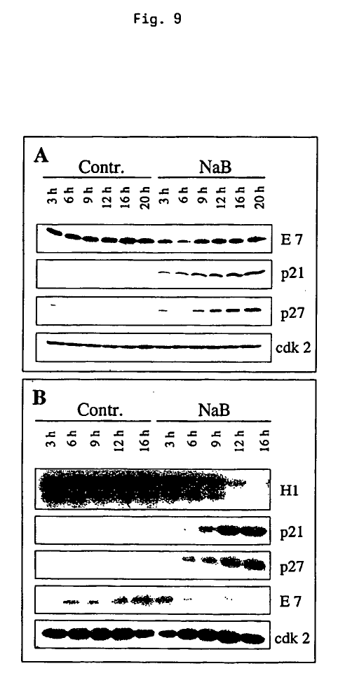

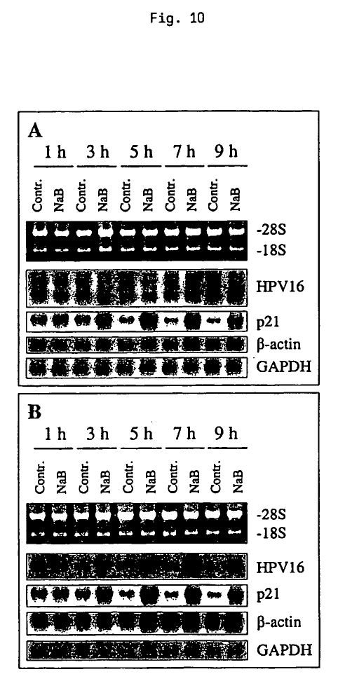

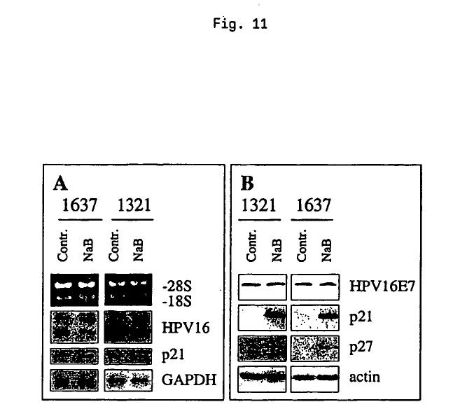

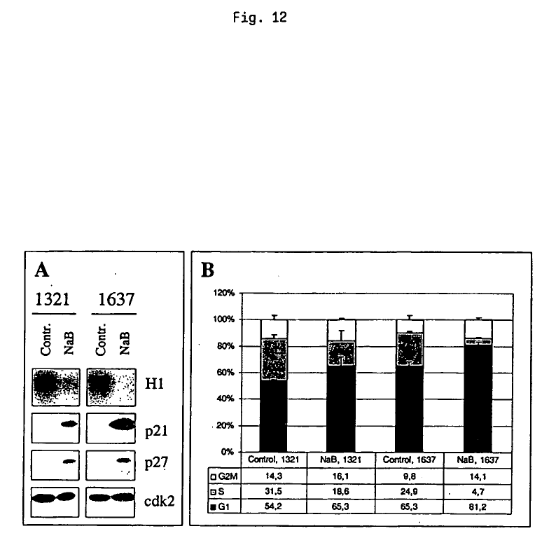

(A and B) Exponentially growing HeLa cells were treated with sodium butyrate (NaB) as indicated in FIG. 1. 50 μg of protein were separated on 12% SDS-PAGE minigels. After electrotransfer, the filters were incubated with antibodies against cyclin D1, cyclin E, cyclin A or cdk2, cdk4 or cdk6 specific antibodies. Equal protein loading was confirmed by reincubating the filters with a monoclonal actin antibody.

(C) Northem blot analysis. 5 μg of RNA were separated in a 1% agarose gel. The filters were hybridized with cDNAs encoding cyclin D1, cyclin E, cyclin A and GAPDH. The positions of the 28S and 18S ribosomal RNA are indicated. (−): untreated cells; (+): treated with 6 mM sodium butyrate for 16 hours.

FIG. 3: Regulation of cyclin-dependent kinase inhibitors and suppression of p45^{SKP2} by sodium butyrate.

(A) Westem blot analysis. 50 μg of cellular protein were loaded on SDS-PAGE minigels. After electrotransfer, the filters were incubated with p21^{CIP1}, p27^{KIP1}, p16^{INK4} and p45^{SKP2}. Equal protein loading was confirmed by a monoclonal actin antibody.

(B) Northem blot analysis. 5 μg of RNA were separated in a 1% agarose gel. Filters were hybridized with cDNAs encoding p21^{CIP1}, p27^{KIP1} and GAPDH. The positions of the 28S and 18S ribosomal RNA are indicated. (−): untreated cells; (+): treated with 6 mM sodium butyrate for 16 hours.

FIG. 4: Sodium butyrate suppresses cdk2 activity by enhanced interaction with p21 and p27 and concomitant loss of E7 binding.

(A) Autoradiography. Cdk2 complexes were immunoprecipitated from HeLa cells and assayed for their activity using histone H1 as substrate. (“P1”: preimmune serum).

(B-E) Western blot analysis of the cdk2 complex. Cdk2 precipitates were separated in a 12% SDS-PAGE gel and immunoblotted with p21^{CIP1} (B), p27^{KIP1} (C) and HPV18 E7 specific antibodies.

(D) Equal loading was verified by incubation with cdk2 antibodies.

(E) P1: preimmune serum. Contr.: untreated cells; NaB: treated with 6 mM sodium butyrate for 16 hours.

FIG. 5: Sodium butyrate mediates pRb degradation without affecting the level of E2F-1: induction of apoptosis in HeLa cells

(A) Northern blot analysis. The same filter was consecutively hybridized with a pRB and GAPDH-specific cDNA. The positions of the 28S and 18S ribosomal RNA in the ethidium bromide stained agarose gel are indicated.

(B and C) Western blot analysis. 50 μg of protein were separated in 8% (for pRb) and 12% SDS-PAGE gels. After electrotransfer, the filters were incubated with pRb, E2F-1 (B) and (C) p53 specific antibodies. Equal protein loading was confirmed with a monoclonal actin antibody.

(D) Quantification of apoptosis using a commercially available “Cell Death Detection ELISA” kit. The enrichment factor, for untreated control cells arbitrarily set as 1, directly reflects the extent of apoptosis in HeLa cells after treatment with sodium butyrate for 16 hours.

FIG. 6: pRB degradation by sodium butyrate in HPV16 immortalized keratinocytes is E7 dependent.

Western blot analysis of cellular extracts obtained from E6-, E7-, and E6/E7-Immortalized cells which were separated in 8% (pRb) and 12% SDS-PAGE gels. After electrotransfer, the filters were incubated with pRb, E2F-1, cyclin E and actin antibodies. (−): untreated cells; (+): treated with 6 mM sodium butyrate for 16 hours. Due to quantitative differences of pRb and cyclin E levels in E7- and E6/E7-positive cells, the filters were exposed for different times.

FIG. 7: Effects of trichostatin A on the expression of cyclins and cyclin-dependent kinase inhibitors and the pRb degradation.

FIG. 8: HPV regulation by sodium butyrate.

(A) HeLa cells were treated with 6 mM sodium butyrate (=NaB) as indicated. (B) HeLa cells were treated with NaB or with 5 μg/ml actinomycin D (=Act D) for different periods of time. For Northern blot analysis, 5 μg of total RNA was separated in 1% agarose gels. The filters were hybridized with probes specific for HPV 18, p21^{CIP1} (=p21), GAPDH or c-myc, respectively. The positions of the 28S and 18S ribosomal RNA are indicated. (Contr.): untreated cells.

FIG. 9: Time-course of cyclin kinase inhibitors and E7 regulation after NaB treatment.

(A) Western blot analysis. 75 μg of total cellular protein was loaded on a 12% SDS-PAGE gel. After electrotransfer, the filters were incubated with antibodies directed against HPV18 E7, p21^{CIP1} (=p21), p27^{KIP1} (=p27) and cdk2.

(B) Cdk2 activity after NaB application. Upper panel: Cdk 2 complexes were immunoprecipitated and assayed for their activity using histone H1 as substrate. Lower panels: Western blot analysis of the cdk 2 complexes which were precipitated, separated in a 12% SDS-PAGE gel and immunoblotted with specific antibodies used in panel A. Loading was verified by incubation with cdk 2 antibodies.

FIG. 10: HPV16 transcription in immortalized keratinocytes after HDAC inhibition.

Keratinocytes immortalized with HPV16 E6/E7 either under control of (A) the homologues viral upstream regulatory region (,,1637″) or (B) of the β-actin promoter (,,1321″) were incubated with NaB for various periods of time. 5 μg of RNA was separated in 1% agarose gels. The filters were hybridized with probes specific for HPV16, p21^{CIP1} (=p21), β-actin and GAPDH. The positions of the 28S and 18S ribosomal RNA are indicated. (Contr.): untreated cells.

FIG. 11: Expression of cell cycle regulatory molecules after HDAC inhibition.

,,1321″ and ,,1637″ cells were treated with 6 mM sodium butyrate for 16 hours.

(A) 5 μg of RNA was separated in 1% agarose gels. The filters were hybridized with HPV 16, p21^{CIP1} (=) p21 and GAPDH-specific probes. The positions of the 28S and 18S ribosomal RNA are indicated.

(B) 75 μg of protein was separated in 12% SDS-PAGE gels as described above (see FIG. 2).

FIG. 12: Cdk2 suppression and cell cycle arrest after NaB treatment. ,,1321″ and ,,1637″ cells were treated with 6 mM sodium butyrate for 16 hours.

(A) Upper panel: cdk2 complexes were immunoprecipitated and assayed for their activity using histone H1 as substrate. Lower panels: Western blot analysis of the cdk 2 complexes. Filters were incubated with specific antibodies against p21^{CIP1} (=p21) and p27^{KIP1} (=p27). Equal precipitation and loading was controlled by incubation with a cdk 2 antibody.

(B) Flow-cytometric analysis. Quantification of the proportion of cells in the cell cycle phases G1, S and G2/M (standard deviations are the mean of three independent experiments). Control: untreated cells; NaB: sodium butyrate 6 mM.

### V. DETAILED DESCRIPTION OF THE INVENTION

It is the object of the present invention to provide means allowing the treatment of diseases associated with an HPV infection.

According to the present invention this is achieved by the subject matter defined in the claims. It has been found during the experiments leading to the present invention that the application of histone deacetylase inhibitors results in the functional inactivation of the viral oncogenes of human papilloviruses (HPV) by reactivation of defense mechanisms of the host cell, leading to growth inhibition and induction of apoptosis of the treated cells. In the experiments it could be shown that the histone deacetylase inhibitors sodium butyrate and trichostatin A arrest human papillomavirus (HPV)-positive cells in G1 to S transition of the cell cycle, which is paralleled by an upregulation of the cyclin dependent kinase inhibitors (CKIs) p21CIP1 and p27KIP1. While these CK1s normally cannot exert their cdk2-inhibitory function in the presence of the viral oncoprotein E7, co-immunoprecipitation experiments revealed that with binding of p21 CIP1 and p27KIP1 to the cyclin-cdk2 complex, E7 binding is prevented. Although HPV expression is thought to be required to maintain a proliferative phenotype of cervical carcinoma cells, exclusion of E7 and complete suppression of cdk2 activity is achieved even in the presence of ongoing viral transcription. Increase of p27KIP1 correlates with downregulation of p45SKP2, a component of the ubiquitin-protein ligase SCFSKP2 which controls the half-life of regulatory proteins during the cell cycle. Besides additional modulatory effects on cyclin expression (cyclin D1 and cyclin A suppression, cyclin E induction), inhibition of histone deacetylation also triggered Rb degradation, while the levels of E2F remained unaffected. The presence of free intracellular E2F and the concomitant induction of p21 and p27 during G1 arrest apparently results in a “conflicting growth situation”, which finally renders the cells to undergo apoptosis. In conclusion, the finding that inhibition of histone deacetylation can bypass the transforming potential of high risk HPV oncoproteins by inducing a block in G1 to S transition and subsequent apoptosis may have important implications for the treatment of HPV mediated diseases like cervical cancer. Since the HDAC inhibitors used in the examples, below, are not toxic for normal cells, it is apparent that these inhibitors are useful for the treatment of diseases associated with an HPV infection.

Accordingly, the present invention relates to the use of a histone deacetylase inhibitor for the preparation of a medicament for the treatment of a disease associated with an HPV infection.

As used herein, the term “histone deacetylase inhibitor” relates to any compound which is capable of inhibiting the activity of histone deacetylase. The person skilled in the art can select suitable compounds on the basis of the known structures (and amino acid sequences) of histone deacetylases, e.g., histone deacetylases 1, 2, 3, 4, 5, 6, 7, 7A, isoform a, 7B, isoform b and 8; see NCBI-Databases AAH00301, XP004370, AAH00614, NP006028, NP005465, NP006035, AAF63491, NP056216, NP057680 and NP060956. Examples of such compounds are antibodies, preferably monoclonal antibodies that specifically react with the histone deacetylase. More recently, WO 98/25146 described further methods for screening libraries of complexes for compounds having a desired property, especially, the capacity to agonize, bind to, or antagonize a polypeptide. The complexes in such libraries comprise a compound under test, a tag recording at least one step in synthesis of the compound, and a tether susceptible to modification by a reporter molecule. Modification of the tether is used to signify that a complex contains a compound having a desired property. The tag can be decoded to reveal at least one step in the synthesis of such a compound. Other methods for identifying compounds which interact with histone deacetylase inhibitors are, for example, the in vitro screening with the phage display system as well as filter binding assays or “real time” measuring of interaction. All these methods can be used to identify inhibitors of histone deacetylases.

Various sources for the basic structure of such an inhibitor can be employed and comprise, for example, mimetic analogs of the histone deacetylase. Mimetic analogs or biologically active fragments thereof can be generated by, for example, substituting the amino acids that are expected to be essential for the biological activity with, e.g., stereoisomers, i.e., D-amino acids; see, e.g., Tsukida, J. Med. Chem. 40 (1997), 3534-3541. Furthermore, in case fragments are used for the design of biologically active analogs pro-mimetic components can be incorporated into a peptide to reestablish at least some of the conformational properties that may have been lost upon removal of part of the original polypeptide; see, e.g., Nachman, Regul. Pept. 57 (1995), 359-370. Furthermore, the histone deacetylase can be used to identify synthetic chemical peptide mimetics that bind to or can function as a ligand, substrate or binding partner of the histone deacetylase as effectively as does the natural polypeptide; see, e.g., Engleman, J. Clin. Invest. 99 (1997), 2284-2292. For example, folding simulations and computer redesign of structural motifs of the histone deacetylase can be performed using appropriate computer programs (Olszewski, Proteins 25 (1996), 286-299; Hoffman, Comput. Appl. Biosci. 11 (1995), 675-679). Computer modeling of protein folding can be used for the conformational and energetic analysis of detailed peptide and protein models (Monge, J. Mol. Biol. 247 (1995), 995-1012; Renouf, Adv. Exp. Med. Biol. 376 (1995), 37-45). In particular, the appropriate programs can be used for the identification of interactive sites of the histone deacetylase and its ligand or other interacting proteins by computer assistant searches for complementary peptide sequences (Fassina, Immunomethods 5 (1994), 114-120). Further appropriate computer systems for the design of protein and peptides are described in the prior art, for example in Berry, Biochem. Soc. Trans. 22 (1994), 1033-1036; Wodak, Ann. N. Y. Acad. Sci. 501 (1987), 1-13; Pabo, Biochemistry 25 (1986), 5987-5991. The results obtained from the above-described computer analysis can be used for, e.g., the preparation of peptide mimetics of a histone deacetylase. Such pseudopeptide analogues of the natural amino acid sequence of the protein may very efficiently mimic the parent protein (Benkirane, J. Biol. Chem. 271 (1996), 33218-33224). For example, incorporation of easily available achiral w-amino acid residues into a histone deacetylase results in the substitution of amide bonds by polymethylene units of an aliphatic chain, thereby providing a convenient strategy for constructing a peptide mimetic (Banerjee, Biopolymers 39 (1996), 769-777). Appropriate peptide mimetics of histone deacetylases can also be identified by the synthesis of peptide mimetic combinatorial libraries through successive amide alkylation and testing the resulting compounds, e.g., for their binding and immunological properties. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, a three-dimensional and/or crystallographic structure of a histone deacetylase can be used for the design of peptide mimetic inhibitors (Rose, Biochemistry 35 (1996), 12933-12944; Rutenber, Bioorg. Med. Chem. 4 (1996), 1545-1558).

Preferred histone deacetylase inhibitors are sodium butyrate, phenylbutyrate and trichostatin A, respectively. Particularly preferred are derivatives of said inhibitors showing increased pharmalogical half-life (Brettman and Chaturvedi, J. Cli. Pharmacol. 36 (1996), 617-622).

The present invention particularly, but not exclusively, relates to the use of a histone deacetylase inhibitor for the preparation of a medicament for the treatment of a disease associated with an infection with an HPV of the HPV-16 and HPV-18 genotypes. The present invention also relates to methods of treatment using the above described histone deacetylase inhibitors. It will be, however, appreciated that the invention extends to variants of such HPV genotypes and other HPV genotypes, e.g HPV1 or HPV11, which may have oncogenic or other pathologic potential.

The uses of the histone deacetylase inhibitors comprise the treatment of any disease associated with an HPV infection, particularly diseases like cervical cancer, cervical intraepithelial neoplasm, wart, larynx papilloma or condyloma acuminatum.

For administration these histone deacetylase inhibitors are preferably combined with suitable pharmaceutical carriers. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Administration of the suitable compositions may be effected by different ways, e.g., by intravenous, intraperetoneal, subcutaneous, intramuscular, topical or intradermal administration. The route of administration, of course, depends on the nature of the disease and the kind of compound contained in the pharmaceutical composition. The dosage regimen will be determined by the attending physician and other clinical factors. As is well known in the medical arts, dosages for any one patient depends on many factors, including the patient's size, body surface area, age, sex, the particular compound to be administered, time and route of administration, the kind of the disease, general health and other drugs being administered concurrently.

The present invention is explained by the examples.

### VI. EXAMPLES

### A. Example 1

### General Methods

1. Cell Lines

HPV18-positive cervical Carcinoma cells (HeLa) were maintained in Dulbecco's modified Eagle's medium (DMEM), supplemented with 10% fetal calf serum (Gibco BRL, Rockville, USA), 1% penicillin and streptomycin (Sigma, Deisenhofen, Deutschland). Primary human keratinocytes, immortalized by E6-, E7- and E6/E7-open reading frames carrying amphotroptic retroviruses were cultivated in “Keratinocyte Medium Kit” (Sigma). Human foreskin keratinocytes transformed with HPV-16 E6/E7 either under the control of a human β-actin promoter (,,1321″) or under the control of the authentic upstream regulatory region HPV (,,1637″) were a kind gift of Dr. R. Schlegel (Georgetown University, Washington, USA) (Münger et al., J. Virol. 63 (1989), 4414-4423; Villa & Schlegel, Virology 181 (1991), 374-377). Cells were grown in 3 parts of keratinocyte-serum free medium (K-SFM, GIBCO, BRL) and 1 part of DMEM containing 10% fetal calf serum (GIBCO, BRL) and 0.75% gentamycin, 0.25% penicillin/streptomycin, respectively

2. Reagents

The sodium salt of n-butyric acid (Sigma) was freshly resolved and diluted with cultivation medium. Trichostatin A (Sigma) was prepared in dimethylsulfoxid (DMSO) (Merck, Darmstadt, Deutschland) Actinomycin D (Sigma) was dissolved in water. 4′,6-Diamidino-2-Phenylindole-2HCl (DAPI) was supplied by SERVA, Heidelberg, Germany and Sulforhodamine 101 (SR101) by Eastman Kodak, Rochester, USA.

3. Cell Cycle Analysis

Cells were harvested by trypsinisation, washed twice with phosphate-buffered saline (PBS) and fixed overnight with 70% ethanol. The cells were resuspended in PBS containing 40 μg/ml of DNase-free RNase A and 50 μg/ml propidium iodide and cell cycle distribution was measured in a fluorescence-activated cell sorter (FACSort) from Becton Dickinson, San Jose, USA. DNA content was quantified by using the “Cell Quest” software (Becton Dickinson, San Jose, USA).

4. RNA Extraction and Northern Blot Analysis

Total cellular RNA was extracted according to the guanidinium-thiocyanate procedure (Chomczynski and Sacchi, Anal. Biochem. 162 (1987), 156-159). Approximately 5 μg RNA were separated on 1% agarose gels in the presence of ethidium bromide under non-denaturing conditions and transferred to GeneScreen Plus membranes (DuPont, Bad Homburg, Deutschland). The filters were hybridized under stringent conditions with specific probes, which were labeled with ³²p-dCTP by random priming (Feinberg and Vogelstein, Anal. Biochem. 137 (1984), 266-267).

5. Hybridization Probes

The cDNAs of p21 (el-Deiry et al., Cell 75 (1993), 817-825), p27 (Polyak et al., Cell 78 (1994), 59-66), cyclin D1 (Baldin et al., Genes Dev. 7 (1993), 812-821), cyclin A (Pines and Hunter, Nature 346 (1990), 760-763) and cyclin E (Hinds et al., Cell 70 (1992), 993-1006) were used. The cDNA stretch of pRB (nucleotide 379-928) was obtained from M. Tommasino (DKFZ Heidelberg). The GAPDH probe (Ercolani et al., J. Biol. Chem. 263 (1988, 15335-15341) was obtained from A. Alonso (DKFZ, Heidelberg). The unit-length HPV18 genome was cloned in pBR322 (Boshart et al., EMBO J. 3 ( 1984), 1151-1157). pHF β-A1 (Gunning et al., Mol. Cell. Biol. 3 (1983), 787-795), harboring an approximately full-length insert of the fibroblast β-actin gene was a generous gift from L. Kedes (Medical Center, Palo Alto, USA). The plasmid c-myc containing the third exon of the human c-myc gene was kindly made available by G. Bornkamm (Institut für Klinische Molekularbiologie, München, Germany) (Polyak et al, Cell 78 (1994), 59-66). The cDNAs for p21^{CIP1} (El-Deiry et al., Cell 75 (1993), 817-825) was kindly provided by B. Vogelstein (John Hopkins Institute, Baltimore, USA) via P. Jansen-Dürr (University of Innsbruck, Austria). The GAPDH probe (Ercolani et al., J. Biol. Chem. 262 (1988), 15335-15341) was provided by A. Alonso (Angewandte Tumorvirologie, DKFZ, Heidelberg). The unit-length HPV 16/18 probes were a kind gift of M. Dürst (Universität Jena, Germany).

6. SDS-PAGE and Western Blots

Cellular extracts were separated in 8-12% SDS PAGE gels and electrotransferred as described elsewhere (Finzer et al., Oncogene 19 (2000), 3235-3244; Soto et al., Oncogene 18 (1999), 3187-3198). The following antibodies were used: cyclin E (HE 12), cyclin D 1 (HD 11), cdk2 (M2), (D-12; Santa Cruz Biotechnology, Inc., Santa Cruz, USA), cdk4 (C-22), cdk6 (C-21), p27^{KIP1} (C-19), (K25020), p45^{SKP2} (N-19), HPV18-E7 (N-19) and E2F-1 (KH95) (Santa Cruz Biotechnology, Inc. Santa Cruz, USA), p21^{CIP1} (C24420; Transduction Laboratories, Lexington, USA), pRB (NCL-RB; Novocastra, UK), p16^{INK4} (15126E, Pharmingen, San Diego, USA) and HPV 16 E7 (ZYMED Lab. Inc., San Francisco, USA). Cyclin A was kindly provided by M. Pagano (Pagano et al., EMBO J. 11 (1992), 961-971). Equal protein transfer and loading was routinely checked by incubating the filters with a monoclonal actin antibody (ICN Biomedicals, Ohio, USA).

7. Extract Preparation, Immunoprecipitation and Histone Kinase Assays

For cell fractionation, cell monolayers were washed twice with PBS and harvested by trypsinisation. Cell extract preparation and cdk2 activity assays were exactly done as described by Blomberg and Hoffmann, Mol. Cell. Biol. 19 (1999), 6183-6194. In addition, cdk2 was immunoprecipitated and analyzed by immunoblotting. Beads used for cdk2 kinase assay were washed 3 times with lysis buffer (Blomberg and Hoffmann, 1999), incubated with Laemmli sample buffer and boiled for 5 min. Supernatant was analyzed by SDS-PAGE gels. Immunoblotting was carried out with the following antibodies: p21^{CIP1} (C24420) and p27^{KIP1} (K25020; Transduction Laboratories) or HPV18-E7 (N-19; Santa Cruz, Inc.). Cdk2 specific antibodies or preimmune serum were kindly provided by I. Hoffmann (DKFZ, Heidelberg).

8. Quantification of Apoptosis

The rate of apoptosis was determined with the Cell Death Detection kit (ELISA^{PLUS}, Roche Diagnostics, Mannheim, Germany) following the instructions of the manufacturer.

9. DNA Staining, Flow Cytometry and Cell Cycle Analysis

Cells were harvested by trypsinisation, washed twice with phosphate-buffered saline (PBS) and fixed overnight in 70% ethanol. After centrifugation, the cell pellet was resuspended in a “DNA/protein staining solution” containing DAPI (5×10⁻⁶ M) and SR 101 (5×10⁻⁶ M) as a protein counter stain following the protocol exactly published by Stoehr et al., Stain Technology 55 (1978), 205-215. Cell cycle analysis and quantification of flow cytometric data was performed according to Dean and Jett, J. Cell. Biol. 60 (1974), 523.

### B. Example 2

### Histone Deacetylase (HDAC) Inhibitors Induce G1/S Phase Arrest in HIPV18-Positive Cervical Carcinoma Cells Despite Ongoing E6/E7 Synthesis

Modulation of histone acetylation is an integral part of a regulatory mechanism, which is involved in the nucleosomal organization of the bulk cellular DNA. Posttranslational neutralisation of the positive charge of lysine residues within the N-terminal domain of core histones relieves histone-DNA interactions, which in turn facilitates the accessibility of transcription factors with their cognate regulatory elements at the DNA level. Alterations of the chromatin architecture is mediated by an interplay between histone acetylases (HAT) and deacetylases (HDAC) counteracting each other in either activating or repressing gene expression. During the last years, a substantial number of HATs have been characterized in yeast and Tetrahymena. In higher eukaryotes, particular adaptor molecules such as p300/CBP or p/CAF (termed as p300/CPB-associated factor) possess intrinsic HAT activity. Their association with CREB, c-jun, c-fos or unliganded nuclear receptors provides a functional linkage between transcriptional co-activators and histone acetylators during initiation of gene expression. Conversely, histone deacetylase type 1 (HDAC1) is an inherent component of a general corepressor complex which interacts with YY-1, Mad/Max as well as the retinoblastoma protein pRb, regularly leading to inhibition of gene expression, although exceptions exist (Workman and Kingston, Annu. Rev. Biochem. 67 (1998), 545-579; Wade et al., Trends Biochem. Sci. 22 (1997), 128-132). Since both HATs and HDACs by themselves have no sequence-specifie DNA-binding affinities, physical interaction with transcriptional activators or repressors provides a reliable explanation by which enzymes, normally acting entirely in a global way during nucleosomal remodelling, can be specified to locally defined transcription units.

Not only cellular transcription factors can target HAT and HDAC molecules, but also viral oncoproteins such as E6 and E7 of the “high-risk” human papillomaviruses (HPV), the etiological agents of cervical cancer. As shown recently, HPV16 E6 is capable of abrogating the costimulatory function of CBP and p300, resulting in a decreased ability of these factors to trans-activate p53-, NF-KB- and c-jun-responsive promoter elements. While E6 interferes with the CBP/p300 tethering function to other transcription factors and possibly with intrinsic HAT activity, E7 oncoprotein can indirectly bind to the histone deacetylase complex via the bridge-protein Mi2β. This property presumably enables E7 to inactivate cellular genes incompatible with the outgrowth of premalignant cells during development of cervical cancer. For example, the interferon-regulatory factor-1 (IRF-1) gene, whose expression is important for interferon signaling and immunological surveillance of persisting HPV infections, is silenced via an E7-mediated recruitment of HDAC to the respective promoter. Depending on the interplay between different transcription factors, E7 can also act in an opposite way. For example, E7 relieves the repressive effect of pRb and HDAC1 on the cyclin E promoter thereby promoting unscheduled cell cycle progression.

Another hallmark of HPV-induced transformation is the post-translational interaction of E6 and E7 with cellular proteins engaged in cell cycle control: E6 binds to p53 and promotes its degradation via the ubiquitin/proteosome pathway. E7 complexes with the retinoblastoma protein pRb, p107, p130, cyclin A, cyclin E as well as the cyclin-dependent kinase inhibitors p21^{CIP1} and p27^{KIP1}.

In order to elucidate the molecular effects of histone deacetylase inhibition in the context of HPV16/18-induced carcinogenesis, the HPV18-positive cervical carcinoma cell line HeLa as well as primary human foreskin keratinocytes, which were separately immortalized with amphotropic retroviruses carrying the open reading frames of HPV16 E6, E7 or E6/E7 were used in the experiments of the present invention. In order to assess the specific impact of histone deacetylase inhibition on cell proliferation, flow cytometric analysis of cellular DNA content was carried out (FIG. 1). Treatment with 6 mM sodium butyrate for 16 hours resulted in a significant increase of the G1 fraction (from 56.8% to 75.7%), whereas the number of cells in S-phase was diminished (from 24.9% to 8.5%). As previously shown, continuous expression of the viral oncogenes seems to be necessary to maintain the proliferative phenotype of cervical carcinoma cells. To address the question whether HDAC inhibitors also have consequences on the transcriptional activity of endogenous HPV18 genomes in HeLa cells, Northern blot analyses were performed. Although time course experiments reproducibly revealed an initial downregulation of viral E6/E7 expression shortly after sodium butyrate addition, the effect was only transient. However, under conditions where cells became growth arrested (FIG. 1A), still ongoing HPV18 transcription (FIG. 1B) and E7 oncogene-expression could be discerned (FIG. 1C). Similar results were obtained with trichostatin A, indicating that HDAC inhibitors in general are able to arrest proliferation of cervical carcinoma cells by circumventing and/or neutralizing viral oncoprotein function.

### C. Example 3

### HDAC Inhibitors Modulate Cyclins but not Cdk Expression

Because the precise mechanism underlying the growth inhibitory effect on cervical carcinoma cells has not yet been established, the steady-state levels of regulatory proteins involved in cell cycle control were examined. As shown in FIG. 2, sodium butyrate selectively downregulates cyclin D1 and cyclin A, while the amount of cyclin E was strongly increased (FIG. 2A). Incubation of the same filter with a monoclonal actin antibody confirmed equal loading and protein transfer. Quantitative differences of cyclin expression after sodium butyrate application were presumably not due to altered degradation rates, since there was a good accordance between the levels of protein and the corresponding mRNAs (FIG. 2C). Control hybridization of the identical blot with glyceraldehyde-3-phosphate dehydrogenase (GAPDH) again corroborated the selectivity of this effect. In contrast, neither the cyclin-dependent kinase 2 (cdk2) nor cdk4 or cdk6 revealed any significant influence on their expression rates (FIG. 2B), clearly excluding the possibility that the growth-inhibitory effect could be attributed to a mere reduction of the intracellular amounts of certain cdks.

### D. Example 4

### Sodium Butyrate Induces Cyclin-Dependent Kinase Inhibitor p21^{CIP1} on Transcriptional Level, While p27^{KIP1} is Upregulated Posttranslationally by Concomitant Suppression of p45^{skp2}, a Component of the Ubiquitin-Protein Ligase SCF^{SKP2}

In a next set of experiments, the levels of cyclin-dependent kinase inhibitors (CKIs), which are known mediators of cell cycle arrest upon various antiproliferative signals were investigated. Western blot analysis of cellular extracts (FIG. 3A) demonstrates that the Cip/Kip family members p21^{CIP1} and p27^{KIP1}, which normally block cyclin A/cdk2 and cyclin E/cdk2 function, were significantly induced after sodium butyrate treatment. In contrast, upon examining the same extracts for the cyclin D1-cdk4/6 inhibitor p16^{INK4}, no reduction was discerned. Surprisingly, monitoring the steady-state levels of the corresponding mRNAs, only p21^{CIP1} was increased, while p27^{KIP1} expression was even diminished under the same experimental conditions. To understand this apparent discrepancy, attention was focused on the expression of p45^{SKP2}, a key regulatory protein recently shown to control the intracellular half-life of p27^{KIP1}. p45^{SKP2} which is found to be upregulated in many transformed cells, became strongly suppressed after addition of sodium butyrate (FIG. 3A). Since the same results were obtained with trichostatin A, these data strongly imply that at least one major cell growth inhibitory function of histone deacetylase inhibitors can be attributed to the post-translational stabilization of the cdk2 inhibitor p27^{KIP1} via disturbance of the SCF (SKP-1-CDC53-F-box) ubiquitin protein ligase complex.

### E. Example 5

### Cdk2 Activity Is Completely Suppressed After Butyrate Treatment: Exclusion of E7 From the Complex

Cdk2 inhibitors such as p21^{CIP1} and p27^{KIP1} play an important role during immortalization and cellular transformation by potential DNA tumorviruses, because their function can be neutralized, or bypassed after binding of viral oncoproteins such as E7. To analyze whether cdk2 activity was modulated after addition of sodium butyrate, cyclin-cdk2 complexes were first immunoprecipitated using specific antibodies directed against cdk2, and subsequently assayed in vitro using histone H1 as substrate (FIG. 4A). In comparison with the untreated control, cdk2 kinase activity was completely abolished 16 hours after sodium butyrate addition. Time course experiments performed in parallel showed that cdk2 was still active after 9 hours, but abruptly declined 3 hours later. To test whether or not p21^{CIP1} and p27^{KIP1} in fact bind to the cdk2 complex, the composition pattern of the cdk2 immunoprecipitates was examined by Western blot analysis. As depicted in FIG. 4B and C, cdk2 activity was abrogated under conditions where both CK1s became associated with the cdk2 complex. In contrast, E7 that was readily detectable in untreated control cells, completely disappeared in the treated cells though the total intracellular net amount of the viral oncoprotein did not change (FIG. 4D), see also FIG. 1C, for comparison). Reincubation of the same immunoblots with a cdk2 antibody confirmed that equal amounts were precipitated (FIG. 4E). Taken together, these data indicate that transition from a proliferative to a quiescent phenotype apparently requires at least two steps: functional abolition of cdk2 activity by p21^{CIP1} and p27^{KIP1} as well as concomitant prevention of E7 binding.

### F. Example 6

### Differential pRB Activation in the Presence of Individual Oncoproteins: the Role of Histone Deacetylase Inhibitors in the Induction of Apoptosis

The most studied G1 specific cyclin/cdk substrate is the retinoblastoma protein pRb, which can recruit HDAC1 to repress cell cycle regulatory proteins such as cyclin E. Since cyclin E was significantly upregulated by sodium butyrate, it was mandatory to examine the fate of pRb in the experimental cell systems. FIG. 5B illustrates that pRB became totally degraded after 16 hours, but histone deacetylase inhibition apparently has no consequences on gene expression and translation of the transcription factor E2F-1. Instead, the absence of pRb could be clearly attributed to a post-translational event, because the steady-state level of the corresponding mRNA was maintained (FIG. 5A). The lack of a modulatory effect on p53 (FIG. 5C), whose half-life is controlled by E6, confirmed the previous finding that viral oncoprotein expression is sustained in the presence of sodium butyrate (see FIG. 1C).

The strong degradation of the anti-apoptotic protein pRb without any reduction of the transcription factor E2F-1 presumably accounts for the final appearance of the distinct apoptotic figures such as membrane blebbing, detachment from the surface and karyorhexis after longer HDAC inhibition. The start of apoptosis can already be detected after 16 hours using a sensitive ELISA assay, which measures the release of histone-associated-DNA-fragments in the cytoplasm after nuclear breakdown. As shown in FIG. 5D, both sodium butyrate (and much stronger trichostatin A) induce programmed cell death to a significant extent.

While E2F-1 also did not significantly vary in HPV16-immortalized human keratinocytes, pRb degradation seems to be strictly dependent on the presence of E7. As shown in FIG. 6, pRb completely disappeared exclusively in E7-expressing cells, while it is only seen to be hypophosphorylated in cells containing E6 as viral oncogene. E6/E7-immortalized keratinocytes again revealed pRb degradation, clearly showing that the fate of pRb is determined by E7 in a dominant fashion. Biological availability of pRb can be followed by monitoring the expression level of cyclin E, whose transcription is negatively regulated by pRb. The reason for cyclin E expression in untreated E7-positive cells is consistent with the ability of E7 to overcome the pRb suppressive effect on the cognate promoter by destroying the pRb-HDACI complex.

### G. Example 7

### Effects of Trichostatin A on the Expression of Cyclins and Cyclin-Dependent Kinase Inhibitors and pRB Degradation

The effects of trichostatin A are shown in FIG. 7(a)-(i)

1. Trichostatin A Downregulates Cyclin A and Upregulates Cyclin E

Exponentially growing HeLa cells were treated with trichostatin A as indicated in FIG. 1. 50 μg of protein was separated on 12% SDS-PAGE minigels. After electrotransfer, the filters were incubated with antibodies against cyclin E and cyclin A. Equal protein loading was confirmed by reincubating the filters with a monoclonal actin antibody. (Contr.): untreated cells; (DMSO): DMSO control; (TSA): treated with 330 nM trichostatin A for 16 hours.

2. Trichostatin A Induces Cyclin-Dependent Kinase Inhibitors P21^{CIP1}, Upregulates P27^{KIP1} and Suppresses P45^{SKP2}

Western blot analysis. 50 μg of cellular protein was loaded on SDS-PAGE minigels. After electrotransfer, the filters were incubated with p21^{CIP1}, p27^{KIP1}, p16^{INK4} and p45^{SKP2}. Equal protein loading was confirmed by a monoclonal actin antibody. (Contr.): untreated cells; (DMSO): DMSO control; (TSA): treated with 330 nM trichostatin A for 16 hours.

4. Trichostatin A Suppresses Cdk2 Activity by Enhanced Interaction with P21^{CIP1} and P27KIP¹ and Concomittant Loss of E7 Binding

Autoradiography. Cdk2 complexes were immunoprecipitated from HeLa cells and assayed for their activity using histone H1 as substrate. (“PI”: preimmune serum).

Western blot analysis of the cdk2 complex. Cdk2 precipitates were separated in a 12% SDS-PAGE gel and immunoblotted with p21^{CIP1}, p27^{KIP1} and HPV 18 E7 specific antibodies. Equal loading was verified by incubation with cdk2 antibodies. PI: preimmune serum. (Contr.): untreated cells; (DMSO): DMSO control; (TSA): treated with 330 nM trichostatin A for 16 hours.

5. Time Course of Cdk2 Suppression After Treatment With Sodium Butyrate

Autoradiography. Cdk2 complexes were immunoprecipitated from HeLa cells and assayed for their activity as in (c). Cells were treated for 3, 6, 9, 12 and 16 hours with 6 mM sodium butyrate (NaB). (Contr.): untreated cells harvested after 3, 6, 9, 12, and 16 hours.

6. Trichostatin A Mediates pRB Degradation without Affecting the Level of E2F-1: Induction of Apoptosis in Hela Cells

Western blot analysis: 50 μg of protein was separated in 8% (for pRb) and 12% SDS-PAGE gels. After electrotransfer, the filters were incubated with pRb, E2F-1 (f) and (g) p53 specific antibodies. Equal protein loading was confirmed with a monoclonal actin antibody.

Quantification of apoptosis using a commercially available “Cell Death Detection ELISA” kit. The enrichment factor, for untreated control cells arbitrarily set as 1, directly reflects the extent of apoptosis in HeLa cells after treatment with trichostatin A for 16 hours. (Contr.): untreated cells; (DMSO): DMSO control; (TSA): treated with 330 nM trichostatin A for 16 hours.

7. Detection of HPV 16 E6 and E7 mRNAs by RT-PCR in the Immortalized Keratinocytes

The expression of E6 and E7 was detected after electrophoresis of the reverse transcriptase (RT)-PCR products on 2% agarose gels. Expected E6, E6* or E7 fragments are indicated. As control GAPDH mRNA was detected by RT-PCR (460 bp). The RT-reaction was performed with the SuperScript TMII (Gibco) following the attached instructions of the manufacturer, using 1.5 μg of total RNA. For E6 detection, the following primers were used: upper primer 5′ ACT GCA ATG TTT CAG GAC CC 3′, lower primer 5′ TCA GGA CAC AGT GGC TTT TG 3′, for the E7 detection; upper primer 5′ CCC AGC TGT AAT CAT GCA TG 3′, lower primer 5′ TGC CCA TTA ACA GGT CTT CC 3′. For GAPDH detection, the following forward (TGG ATA TTG TTG CCA TCA ATG ACC) and reverse (GAT GGC ATG GAC TGT GGT CAT G) primers were used. Conditions for all sets of primers were: initially denaturation time of 3 min. at 94° C. and then 30 sec. at 94° C., 1 min. at 60° C., 1 min. at 72° C. (35 cycles) and 10 min. at 72° C. (1): E6 and (2) E6* fragments; (#): E7 fragment; (+): treated with 6 mM sodium butyrate for 16 hours; (−): untreated cells.

### H. Example 8

### Growth Arrest of HPV-Positive Cells After Histone Deacetylase Inhibition is Independent of E6/E7 Oncogene Expression

1. Inhibitors of Histone Deacetylase Transiently Downregulate HPV Transcription

Carrying out time-course experiments of HPV18 in the presence of 6 mM NaB (FIG. 8A), viral transcription was found to be selectively, but transiently downregulated. Starting at about three hours, strongest suppression could be discerned at six hours, reaching back to steady state levels (after sixteen hours) initially detected in the untreated controls. On the contrary, considering the cyclin-dependent kinase inhibitor p21^{CIP1}, the corresponding transcript was already elevated after 3 hours and remained induced during the whole incubation period. Subsequent hybridization of the same filter with a housekeeping gene not affected by HDAC inhibition, glyceraldehyd-3-phosphate dehydrogenase (GAPDH) mRNA demonstrated that transient suppression was in fact selective directed against the virus-specific transcription cassette and did not represent the result of a non-specific impairment of total cell transcription. It should be stressed that similar kinetics were also obtained with trichostatin A (data not shown), indicating that transient HPV downregulation is a general feature induced by HDAC inhibitors (see also FIG. 10A). Nonetheless, despite viral transcription re-appeared, cdk2 activity was suppressed and the cells finally became growth arrested (see FIG. 9A).

If transient downregulation of HPV18 expression was the result of a mechanism regulated at the level of initiation of transcription, one can anticipate that the rate of mRNA decay after NaB application should follow roughly a similar kinetic as with actinomycin D, known to non-specifically block RNA polymerase transcription by intercalating into the DNA. To get insight in this question, cells were separately treated either with 5 μg/ml actinomycin D or with NaB for different time intervals (1, 2, 3, 4, 5 hours, respectively) and the RNA was examined by Northern blot analysis. As depicted in FIG. 8B, the relative abundance of the HPV-specific transcripts began to decrease at roughly the same time range (between two-three hours) independently of which inhibitor was applied. These data are in agreement with preliminary nuclear run-on analyses, where nascent HPV18 transcripts were also selectively diminished (data not shown). Since viral RNA, however, reproducibly dropped more sharply three hours after NaB (and TSA) treatment when compared with actinomycin D, an additional post-transcriptional regulation cannot be excluded at the moment. Expression of the c-myc gene, whose RNA is extremely unstable both in normal and tumor cells was used as an further reference to assess the biological activity of actinomycin D under our experimental conditions. Finally, it should be noted that, in contrast to a recent report using oxamflatin as HDAC inhibitor (Kim et al., Oncogene 18 (1999), 2461-2470), de novo protein synthesis was not required to abolish the biological effect of NaB or TSA on HPV-positive cells, independently of whether a simultaneous or consecutive incubation with cycloheximide was carried out (data not shown).

2. Time-Course Analysis of Viral and Cell Cycle Regulatory Proteins After HDAC Inhibition

Since it is supposed that the blockage of the growth inhibitory function of cyclin-dependent kinase inhibitors (CKIs) through E7 binding represents a key regulatory event during the development of cervical cancer, time-course expression of p21^{CIP1} and p27^{KIP1} together with E7 was monitored by Western blot analysis. As demonstrated in FIG. 9A, regulation of the mRNA (FIG. 8A) closely correlates with the amount of the corresponding protein, showing a significant reduction of the E7 oncoprotein six hours after NaB application. However, there is apparently no causal link between the time range of HPV suppression and the extent of p21^{CIP1} and p27^{KIP1} protein levels, because both CKIs reach their plateau under conditions, when viral RNA became fully re-expressed (compare FIGS. 8A and 9A). The amount of cyclin-dependent kinase 2 (cdk2) was completely unaffected by NaB treatment.

To analyse the temporal range of cdk2 activity suppression, cyclin-cdk2 complexes were first immunoprecipitated with a cdk2 specific antibody and subsequently functionally tested in an in vitro phosphorylation assay using histone H1 as substrate (FIG. 9B, upper panel). When compared with untreated controls, cdk2 remained active up to 9 hours, but immediately declined between 12-16 hours after NaB addition. To examine the association kinetics of p21^{CIP1} and p27^{KIP1}, the composition pattern of the cdk2 immunoprecipitates was examined by Western blot analysis. As depicted in FIG. 9B, cdk2 activity was suppressed under conditions where threshold amounts of both CKIs became associated with the cdk2 complex (between 12-16 hours). Conversely, E7 bound to cyclin-cdk2 complexes in control cells completely disappeared between 12-16 hours despite ongoing viral RNA production (FIG. 8A) and re-synthesized intracellular E7 to quantities comparable to untreated cells (FIG. 9B, see also FIG. 9A, for comparison). Incubation of the same filter with a cdk2 specific antibody confirmed that approximately equal amounts were precipitated.

3. Cdk2 Inhibition and Growth Arrest in HPV16 Immortalized Keratinocytes Expressing the Viral Oncogenes under the Control of a Heterologous Promoter

Since previous studies claimed that continuous expression of the viral oncogenes is indispensable to sustain proliferation of cervical carcinoma cells both in vitro and in vivo there still exists the possibility that transitory suppression of HPV transcription is a precondition to allow growth arrest via HDAC inhibition. In order to analyse this issue in molecular terms, advantage was taken of two immortalized keratinocyte cell lines where HPV16 E6/E7 was either under control the HPV16 upstream regulatory region (URR) (designated as ,,1637″) or directed by a heterologous promoter derived from the human β-actin gene (referred as ,,1321″) (Münger et al., 1989, supra; Villa & Schlegel, 1991, supra). While p21CIP1 expression was augmented in both cell lines six hours following NaB addition, time course of transient HPV16 mRNA suppression in ,,1637″ cells (FIG. 10A) was similar to that previously observed in HPV18-positive HeLa cells (see also FIG. 8A). In contrast, a strong upregulation of HPV could be discerned in the ,,1321″ cell line, where viral transcription became induced three hours after treatment and remained elevated during the entire incubation period used (FIG. 10B). Increased β-actin promotor directed HPV16 expression was not the consequence of a position effect after transfection, since the same was obtained when endogeneous β-actin expression was examined (compare FIGS. 10A and 10B). After longer incubation (16 hours), HPV mRNA declined to basal levels initially detected in untreated cells (FIG. 11A). Transcription was paralleled by protein re-expression, showing equal amounts of HPV 16 E7 16 hours after NaB application when compared with the corresponding controls (FIG. 11B). Analogous results were obtained with TSA indicating that divergent HPV regulation was not a peculiarity of NaB (data not shown). In any case, as already depicted for HeLa cells (FIG. 9A), both cdk2 inhibitors p21CIP1 and p27KIP1 were increased to the same extent independently of whether viral oncogene expression was directed by the HPV16-URR or by the β-actin promoter (FIG. 11A). Incubations of identical filters with a monoclonal actin antibody again confirmed equal loading and protein transfer.

As next, it was set out to determine the function of the cdk2 complex itself, since it could be conceivable that the elevated—instead of decreased—level of HPV 16 expression in ,,1321″ immortalized keratinocytes failed to block cdk2 activity despite cyclin-dependent kinase inhibitors were accumulated. However, monitoring cdk2 function after immunoprecipitation, both complexes revealed high amounts of co-precipitated, and therefore physically linked p21^{CIP1} and p27^{KIP1},which account for the inability of cdk2 to phosphorylate histone H1 as substrate when assayed after 16 hours (FIG. 12A). Abrogation of cdk 2 activity properly correlate with cell cycle data obtained after flow cytometry, where a significant accumulation of cells in G1 (from 54.2% to 65.3% for ,,1321″ cells and from 65.3% to 81.2% for ,,1621″ keratinocytes) could be noticed. Conversely, the proportion of cells within S-phase was significantly decreased (31.5% to 18.6% for ,,1321″ and from 24.9% to 4.7% for ,,1621″, respectively) (FIG. 12B). These results demonstrate that HPV16/18-positive cells can be growth arrested by HDAC inhibitors despite ongoing HPV transcription and thus independently from any potential position effects uncoupling URR-directed gene expression by adjacent cellular promoters or by downstream 3′-polyadenylation sites after viral integration into the host genome during multi-step carcinogenesis.

What is claimed is:

1. Use of a histone deacetylase inhibitor for the preparation of a medicament for the treatment of a disease associated with an HPV infection.

2. Use according to claim 1, wherein the HPV infection is a HPV1, HPV11, HPV16 or HPV18 infection.

3. Use according to claim 1 or 2, wherein the disease is cervical cancer, cervical intraepithelial neoplasm, wart, larynx papilloma or condyloma acuminatum.

4. Use according to any one of claims 1 to 3, wherein the histone deacetylase inhibitor is sodium butyrate, phenylbutyrate or trichostatin A.
